(19) United States
(12) Patent Application Publication
(10) Pub. No.: US 2021/0128003 A1
(43) Pub. Date: May 6, 2021

(54) LONG-TERM HEARTRATE TRENDS

(71) Applicant: West Affum Holdings Corp., Grand Cayman (KY)

(72) Inventors: Kelly A. Brennan, Renton, WA (US); Joseph L. Sullivan, Kirkland, WA (US); Laura M. Gustavson, Redmond, WA (US)

(73) Assignee: West Affum Holdings Corp., Grand Cayman (KY)

(21) Appl. No.: 17/087,044

(22) Filed: Nov. 2, 2020

Related U.S. Application Data
(60) Provisional application No. 62/929,789, filed on Nov. 2, 2019.

(51) Int. Cl. A61B 5/044; A61B 5/0472; A61B 5/0452; A61N 1/39

(52) U.S. Cl. CPC A61B 5/044; A61B 5/0472; A61N 1/3968; A61N 1/3904; A61B 5/04525

(57) ABSTRACT

In one embodiment, a method to display data collected by a wearable cardioverter defibrillator (WCD) is described. The method includes receiving raw heart rate data from one or more electrocardiogram (ECG) sensors. The method also includes classifying the heart rate data into one of a ventricular (VT) and supra-ventricular (SVT) segments based at least in part on the QRS width, recording the classification and time stamp of each VT and SVT heart rate, and generating one or more interactive displays of the VT and SVT segments.

### CROSS REFERENCE TO RELATED PATENT APPLICATIONS

This patent application claims benefit of U.S. Provisional Patent Application No. 62/929,789 filed Nov. 2, 2019 and is incorporated herein by reference in their entirety for all purposes.

### BACKGROUND

When people suffer from some types of heart arrhythmias, in some instances, blood flow to various parts of the body may be reduced. Some arrhythmias can result in a Sudden Cardiac Arrest (SCA). SCA can lead to death very quickly, e.g. within 10 minutes, unless treated in the interim. Some observers have thought that SCA is the same as a heart attack, which it is not.

Some people have an increased risk of SCA. Such people may include patients who have had a heart attack or a prior SCA episode. A frequent recommendation for these people is to receive an Implantable Cardioverter Defibrillator (ICD). The ICD is surgically implanted in the chest, and continuously monitors the patient's intracardiac electrogram (IEGM). If certain types of heart arrhythmias are detected, then the ICD delivers an electric shock through the heart.

As a further precaution, people who have been identified to have an increased risk of a SCA are sometimes given a Wearable Cardioverter Defibrillator (WCD) system to wear until an ICD is implanted. Early versions of such systems were called wearable cardiac defibrillator systems. A WCD system typically includes a harness, vest, belt, or other garment that the patient wears. The WCD system further includes electronic components, such as a defibrillator and electrodes, coupled to the harness, vest, or another garment. When the patient wears the WCD system, the electrodes may electrically contact the patient's skin, and aid in sensing the patient's electrocardiogram (ECG). If a shockable heart arrhythmia (e.g., ventricular fibrillation or VF) is detected from the ECG, then the defibrillator delivers an appropriate electric shock through the patient's body, and thus through the heart. The delivered shock may restart the patient's heart and save the patient's life.

### BRIEF SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure describes instances and examples of cardiac monitoring systems (e.g., WCD systems), devices, systems, storage media that may store programs, and methods. In one embodiment, a method to display data collected by a wearable cardioverter defibrillator (WCD) is described. The method includes receiving raw heart rate data from one or more electrocardiogram (ECG) sensors. The method also includes classifying the segment data into one of a ventricular (VT) and supra-ventricular (SVT) segments based at least in part on the QRS width, recording the classification and time stamp of each VT and SVT segment, and generating one or more interactive displays of the VT and SVT segments.

In one embodiment, classifying the segment data may include generating a template for a normal QRS heart rate complex for a user of the WCD and comparing the template to the QRS width of the raw heart rate data. In another embodiment, the method may include generating a graph of VT and SVT segments displaying a QRS width of each VT and SVT beat. In another embodiment, the method may include determining at least one of a maximum, a minimum, an average, and some combination thereof of the VT and SVT segments once every predetermined time period. In some embodiments, the method may include analyzing a QRS width of the raw heart rate data.

In another embodiment, the method may determine a percentage of beats that are VT beats and a percentage of beats that are SVT beats for a predetermined time period and generate a visual representation to visibly display the percentage of VT beats and SVT beats for the predetermined time period. In some embodiments, the predetermined time period may be adjustable.

In some embodiments, the method may generate a graph of VT beats per minute for a predetermined time period and generate a graph of SVT beats per minute for the predetermined time period. The graphs may be combined into one or more displays. In some embodiments, the method may include receiving movement data from a motion sensor coupled to the WCD, analyzing movement data for patient mobility data, generating a graph of the patient mobility data, and displaying the graph of patient mobility data with the VT and SVT segments. In some embodiments, the method may generate a maximum, minimum, and average segment reading for a specific time stamp and display the maximum, minimum, and average segment for the specific time stamp when prompted. In some embodiments, the interactive display may be one of a line chart, bar chart, pie chart, and histogram.

In one embodiment, a wearable cardiac defibrillator (WCD) system for monitoring health of a patient wearing the WCD system is described. The WCD includes at least one sensor positioned to gather data about the patient, one or more memories, the one or more memories configured to analyze patient data, and one or more processors. The processor is configured receive raw segment data from one or more electrocardiogram (ECG) sensors and analyze a QRS width of the raw segment data. The processor is further configured to classify the segment data into one of a ventricular (VT) and supra-ventricular (SVT) heartbeat based at least in part on the QRS width and record the classification and time stamp of each VT and SVT segment. The processor then generates an interactive display of the VT and SVT segments.

In some embodiments, classifying the segment data, the processor may generate a template for a normal QRS heart rate complex for a user of the WCD and comparing the template to the QRS width of the raw heart rate data. In some embodiments, the processor may generate one or more graphs of VT and SVT heart beats displaying a QRS width of each VT and SVT beat. In one embodiment, the processor may determine at least one of a maximum, a minimum, an average, and some combination thereof of the VT and SVT segment once every predetermined time period.

In some embodiments, the processor may determine a percentage of beats that are VT beats and a percentage of beats that are SVT beats for a predetermined time period and generate a visual representation to visibly display the percentage of VT beats and SVT beats for the predetermined time period. In some embodiments, the predetermined time period may be adjustable. In some embodiments, the processor may generate a graph of VT beats per minute for a predetermined time period, generate a graph of SVT beats per minute for the predetermined time period, and combine the graphs into a single.

In some embodiments, the processor may receive movement data from a motion sensor coupled to the WCD and analyze movement data for patient mobility data. The processor may also generate a graph of the patient mobility data and display the graph of patient mobility data with the VT and SVT heart rates. In some embodiments, the processor may determine a maximum, minimum, and average heart rate reading for a specific time stamp and display the maximum, minimum, and average heart rate for the specific time stamp when prompted.

In a further embodiment, a method to display data collected by a wearable cardioverter defibrillator is described. The method includes positioning at least one electrocardiogram (ECG) sensing electrodes to measure electrical activity of a heart of a person and receiving at least one ECG signal from the at least one ECG electrodes. The method analyzes the signal into usable data including a QRS width of each heartbeat and classifies the segment data into one of a ventricular (VT) and supra-ventricular (SVT) segment based at least in part on the QRS width. The method records the classification and time stamp of each VT and SVT segment and generates an interactive display of the VT and SVT segments.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a diagram of a sample WCD system in accordance with exemplary embodiments described herein;

FIG. 2 is a block diagram of an example defibrillator in accordance with exemplary embodiments described herein;

FIG. 3 is a diagram of sample embodiments of components of a WCD system in accordance with exemplary embodiments described herein;

FIG. 4 is a is a block diagram of an example defibrillator in accordance with exemplary embodiments described herein;

FIG. 5 is an exemplary flow diagram in accordance with exemplary embodiments described herein;

FIG. 6 is another exemplary flow diagram in accordance with exemplary embodiments described herein;

FIG. 7 is an exemplary graphical representation of user data in accordance with exemplary embodiments described herein;

FIG. 8 is another exemplary graphical representation of user data in accordance with exemplary embodiments described herein;

FIG. 9 is another exemplary graphical representation of user data in accordance with exemplary embodiments described herein; and

FIG. 10 is another exemplary graphical representation of user data in accordance with exemplary embodiments described herein.

### DETAILED DESCRIPTION

The detailed description set forth below in connection with the appended drawings, where like numerals reference like elements, are intended as a description of various embodiments of the present disclosure and are not intended to represent the only embodiments. Each embodiment described in this disclosure is provided merely as an example or illustration and should not be construed as precluding other embodiments. The illustrative examples provided herein are not intended to be exhaustive or to limit the disclosure to the precise forms disclosed.

In the following description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

Wearable Cardioverter Defibrillators (WCDs) are worn by patients at risk for sudden cardiac arrest. Knowing the heart rate trends of patients is useful for attending physicians to provide an understanding of the patient's condition over a long duration of time. However, knowing general heart rate trends may not always indicate if the patient is experiencing ventricular tachycardia (VT) or supra-ventricular tachycardia (SVT). VT is a fast, abnormal heart rate. A VT heartbeat starts in the heart's lower chambers, called the ventricles. VT is defined as three or more heartbeats in a row, at a rate of more than 100 beats a minute. If VT lasts for more than a few seconds at a time, it may become life-threatening. SVT is as an abnormally fast heartbeat. SVT can include many forms of heart rhythm problems, including heart arrhythmias, that originate above the ventricles (supraventricular) in the atria node. Different rhythms may have different implications for the patient which may be better treated by the physician if more information is available to treat the patient other than a simple heartbeat categorization. Therefore, the patient and the physician may benefit from knowing the type of rhythms present, the duration of the particular rhythm, and when it was present.

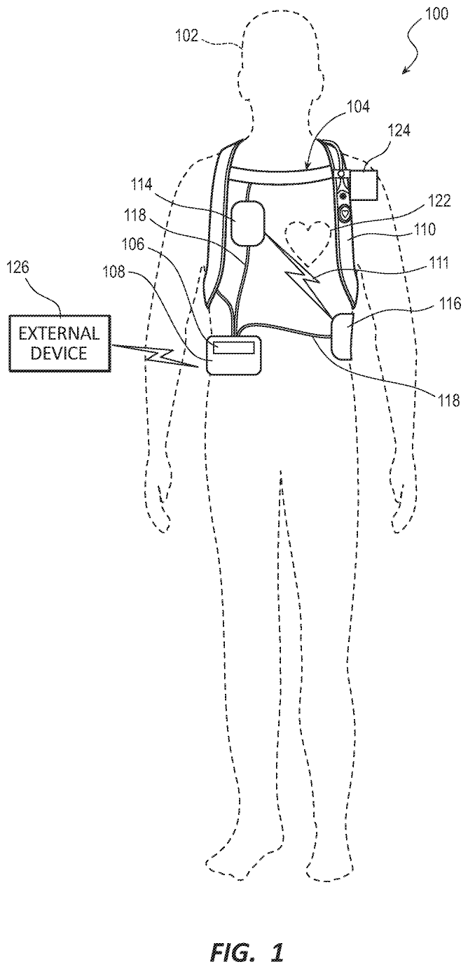

FIG. 1 illustrates a system 100 with a patient 102 wearing an example of a WCD system 104 according to embodiments described herein. In some embodiments, the WCD system 104 may include one or more communication devices 106, a support structure 110, and an external defibrillator 108 connected to two or more defibrillation electrodes 114, 116, among other components.

The support structure 110 may be worn by the patient 102. The patient 102 may be ambulatory, meaning the patient 102 can walk around and is not necessarily bed-ridden while wearing the wearable portion of the WCD system 104. While the patient 102 may be considered a “user” of the WCD system 104, this is not a requirement. For instance, a user of the WCD system 104 may also be a clinician such as a doctor, nurse, emergency medical technician (EMT) or other similarly tasked individual or group of individuals. In some cases, a user may even be a bystander. The particular context of these and other related terms within this description should be interpreted accordingly.

In some embodiments, the support structure 110 may include a vest, shirt, series of straps, or other system enabling the patient 102 to carry at least a portion of the WCD system 104 on the patient's body. In some embodiments, the support structure 110 may comprise a single component. For example, the support structure 110 may comprise a vest or shirt that properly locates the WCD system 104 on a torso 112 of the patient 102. The single component of the support structure 110 may additionally carry or couple to all of the various components of the WCD system 104.

In other embodiments, the support structure 110 may comprise multiple components. For example, the support structure 110 may include a first component resting on a patient's shoulders. The first component may properly locate a series of defibrillation electrodes 114, 116 on the torso 112 of the patient 102. A second component may rest more towards a patient's hips, whereby the second component may be positioned such that the patient's hips support the heavier components of the WCD system 104. In some embodiments, the heavier components of the WCD system 104 may be carried via a shoulder strap or may be kept close to the patient 102 such as in a cart, bag, stroller, wheelchair, or other vehicle.

The external defibrillator 108 may be coupled to the support structure 110 or may be carried remotely from the patient 102. The external defibrillator 108 may be triggered to deliver an electric shock to the patient 102 when patient 102 wears the WCD system 104. For example, if certain thresholds are exceeded or met, the external defibrillator 108 may engage and deliver a shock to the patient 102.

The defibrillation electrodes 114, 116 can be configured to be worn by patient 102 in a number of ways. For instance, the defibrillator 108 and the defibrillation electrodes 114, 116 can be coupled to the support structure 110 directly or indirectly. For example, the support structure 110 can be configured to be worn by the patient 102 to maintain at least one of the electrodes 114, 116 on the body of the patient 102, while the patient 102 is moving around, etc. The electrodes 114, 116 can be thus maintained on the torso 112 by being attached to the skin of patient 102, simply pressed against the skin directly or through garments, etc. In some embodiments, the electrodes 114, 116 are not necessarily pressed against the skin but becomes biased that way upon sensing a condition that could merit intervention by the WCD system 104. In addition, many of the components of defibrillator 108 can be considered coupled to support structure 110 directly, or indirectly via at least one of defibrillation electrodes 114, 116.

The WCD system 104 may defibrillate the patient 102 by delivering an electrical charge, pulse, or shock 111 to the patient 102 through a series of electrodes 114, 116 positioned on the torso 112. For example, when defibrillation electrodes 114, 116 are in good electrical contact with the torso 112 of patient 102, the defibrillator 108 can administer, via electrodes 114, 116, a brief, strong electric pulse 111 through the body. The pulse 111 is also known as shock, defibrillation shock, therapy, electrotherapy, therapy shock, etc. The pulse 111 is intended to go through and restart heart 122, in an effort to save the life of patient 102. The pulse 111 can further include one or more pacing pulses of lesser magnitude to pace heart 122 if needed. The electrodes 114, 116 may be electrically coupled to the external defibrillator 108 via a series of electrode leads 118. The defibrillator 108 may administer an electric shock 111 to the body of the patient 102 when the defibrillation electrodes 114, 116 are in good electrical contact with the torso 112 of patient 102. In some embodiments, devices (not shown) proximate the electrodes 114, 116 may emit a conductive fluid to encourage electrical contact between the patient 102 and the electrodes 114, 116.

In some embodiments, the WCD system 104 may also include either an external or internal monitoring device or some combination thereof. FIG. 1 displays an external monitoring device 124 which may also be known as an outside monitoring device. The monitoring device 124 may monitor at least one local parameter. Local parameters may include a physical state of the patient 102 such as ECG, movement, heartrate, pulse, temperature, and the like. Local parameters may also include a parameter of the WCD 104, environmental parameters, or the like. The monitoring device 124 may be physically coupled to the support structure 110 or may be proximate the support structure 110. In either location, the monitoring device 124 is communicatively coupled with other components of the WCD 104.

For some of these parameters, the device 124 may include one or more sensors or transducers. Each one of such sensors can be configured to sense a parameter of the patient 102, and to render an input responsive to the sensed parameter. In some embodiments, the input is quantitative, such as values of a sensed parameter; in other embodiments, the input is qualitative, such as informing whether or not a threshold is crossed. In some instances, these inputs about the patient 102 are also referred to herein as patient physiological inputs and patient inputs. In some embodiments, a sensor can be construed more broadly, as encompassing many individual sensors.

In some embodiments, a communication device 106 may enable the patient 102 to interact with, and garnish data from, the WCD system 104. The communication device 106 may enable a patient or third party to view patient data, dismiss a shock if the patient is still conscious, turn off an alarm, and otherwise engage with the WCD system 104. In some instances, the communication device 106 may transfer or transmit information include patient data to a third-party data server such as a cloud server or a blockchain server. In some embodiments, the communication device 106 may be a separable part of an external defibrillator 108. For example, the communication device 106 may be a separate device coupled to the external defibrillator 108. In some embodiments, the communication device 106 may be wired or wirelessly linked to the external defibrillator 108 and may be removable from the defibrillator 108. In other embodiments, the communication device 106 may form an inseparable assembly and share internal components with the external defibrillator 108. In some embodiments, the WCD system 104 may include more than one communication device 106. For example, the defibrillator 108 may include components able to communicate to the patient and the WCD system 104 may include a separate communication device 106 remote form the defibrillator 108.

In some embodiments, the defibrillator 108 may connect with one or more external devices 126. For example, as shown in FIG. 1, the defibrillator 108 may connect to various external devices 126 such as a the cloud, a remote desktop, a laptop, a mobile device, or other external device using a network such as the Internet, local area networks, wide area networks, virtual private networks (VPN), other communication networks or channels, or any combination thereof.

In embodiments, one or more of the components of the exemplary WCD system 104 may be customized for the patient 102. Customization may include a number of aspects including, but not limited to, fitting the support structure 110 to the torso 112 of patient 102; baseline physiological parameters of patient 102 can be measured, such as the heart rate of patient 102 while resting, while walking, motion detector outputs while walking, etc. The measured values of such baseline physiological parameters can be used to customize the WCD system, in order to make its diagnoses more accurate, since patients' bodies differ from one another. Of course, such parameter values can be stored in a memory of the WCD system, and the like. Moreover, a programming interface can be made according to embodiments, which receives such measured values of baseline physiological parameters. Such a programming interface may input automatically in the WCD system these, along with other data.

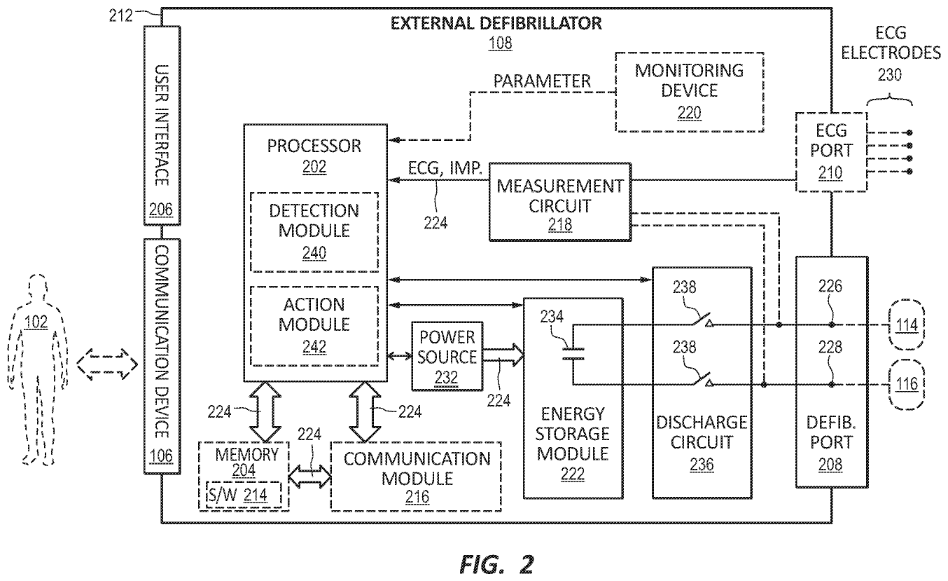

FIG. 2 is a diagram displaying various components of an example external defibrillator 108. The external defibrillator 108 may be an example of the defibrillator 108 described with reference to FIG. 1. The components shown in FIG. 2 may be contained within a single unit or may be separated amongst two or more units in communication with each other. The defibrillator 108 may include a communication device 106, processor 202, memory 204, defibrillation port 208, and ECG port 210, among other components. In some embodiments, the components are contained within a housing 212 or casing. The housing 212 may comprise a hard shell around the components or may comprise a softer shell for increased patient comfort.

The communication device 106, processor 202, memory 204 (including software/firmware code (SW) 214), defibrillation port 208, ECG port 210, communication module 216, measurement circuit 218, monitoring device 220, and energy storage module 222 may communicate, directly or indirectly, with one another via one or more buses 224. The one or more buses 224 may allow data communication between the elements and/or modules of the defibrillator 108.

The memory 204 may include random access memory (RAM), read only memory (ROM), flash RAM, and/or other types. The memory 204 may store computer-readable, computer-executable software/firmware code 214 including instructions that, when executed, cause the processor 202 to perform various functions (e.g., determine shock criteria, determine consciousness of patient, track patient parameters, determine type of heartrates, illustrate heartrate trends, etc.). In some embodiments, the processor 202 may include an intelligent hardware device, e.g., a central processing unit (CPU), a microcontroller, an application-specific integrated circuit (ASIC), etc.

In some embodiments, the memory 204 can contain, among other things, the Basic Input-Output system (BIOS) which may control basic hardware and/or software operations such interactions and workings of the various components of the defibrillator 108, and in some embodiments, components external to the defibrillator 108. For example, the memory 204 may contain various modules to implement the workings of the defibrillator 108 and other aspects of the present disclosure.

In some embodiments, the defibrillator 108 may include a user interface 206. The user interface 406 may be in addition to or part of the communication device 106. The user interface 406 may display an ECG of the patient, a status of the defibrillator 108, a status of a charge (e.g. a battery charge or an energy storage module), and the like.

In some embodiments, the defibrillator 108 may include a defibrillation port 208. The defibrillation port 208 may comprise a socket, opening, or electrical connection in the housing 212. In some instances, the defibrillation port 208 may include two or more nodes 226, 228. The two or more nodes 226, 228 may accept two or more defibrillation electrodes (e.g. defibrillation electrodes 114, 116, FIG. 1). The nodes 226, 228 may provide an electrical connection between the defibrillation electrodes 114, 116 and the defibrillator 108. The defibrillation electrodes 114, 116 may plug into the two or more nodes 226, 228 via one or more leads (e.g. leads 118), or, in some instances, the defibrillation electrodes 114, 116 may be hardwired to the nodes 226, 228. Once an electrical connection is established between the defibrillation port 208 and the electrodes 114, 116, the defibrillator 108 may be able to deliver an electric shock to the patient 102.

In some embodiments, the defibrillator 108 may include an ECG port 210 in the housing 212. The ECG port 210 may accept one or more ECG electrodes 230 or ECG leads. In some instances, the ECG electrodes 230 sense a patient's ECG signal. For example, the ECG electrodes 230 may record electrical activity generated by heart muscle depolarization. The ECG electrodes 230 may utilize 4-leads to 12-leads or multichannel ECG, or the like. The ECG electrodes 230 may connect with the patient's skin.

In some embodiments, the defibrillator 108 may include a measurement circuit 218. The measurement circuit 218 may be in communication with the ECG port 210. For example, the measurement circuit 218 may receive physiological signals from ECG port 210. The measurement circuit 218 may additionally or alternatively receive physiological signals via the defibrillation port 208 when defibrillation electrodes 114, 116 are attached to the patient 102. The measurement circuit 218 may determine a patient's ECG signal from a difference in voltage between the defibrillation electrodes 114, 116.

In some embodiments, the measurement circuit 218 may monitor the electrical connection between the defibrillation electrodes 114, 116 and the skin of the patient 102. For example, the measurement circuit 218 can detect impedance between electrodes 114, 116. The impedance may indicate the effective resistance of an electric circuit. An impedance calculation may determine when the electrodes 114, 116 have a good electrical connection with the patient's body.

In some embodiments, the defibrillator 108 may include an internal monitoring device 220 within the housing 212. The monitoring device 220 may monitor at least one local parameter. Local parameters may include physical state of the patient such as ECG, movement, heartrate, pulse, temperature, and the like. Local parameters may also include a parameter of the WCD system (e.g. WCD 104, FIG. 1), defibrillator 108, environmental parameters, or the like.

In some embodiments, the WCD system 104 may include an internal monitoring device 220 and an external monitoring device (e.g. external monitoring device 124). If both monitoring devices 124, 220 are present, the monitoring devices 124, 220 may work together to parse out specific parameters depending on position, location, and other factors. For example, the external monitoring device 124 may monitor environmental parameters while the internal monitoring device 220 may monitor patient and system parameters.

In some embodiments, the defibrillator 108 may include a power source 232. The power source 232 may comprise a battery or battery pack, which may be rechargeable. In some instances, the power source 232 may comprise a series of different batteries to ensure the defibrillator 108 has power. For example, the power source 232 may include a series of rechargeable batteries as a prime power source and a series of non-rechargeable batteries as a secondary source. If the patient 102 is proximate an AC power source, such as when sitting down, sleeping, or the like, the power source 232 may include an AC override wherein the power source 232 draws power from the AC source.

In some embodiments, the defibrillator 108 may include an energy storage module 222. The energy storage module 222 may store electrical energy in preparation or anticipation of providing a sudden discharge of electrical energy to the patient. In some embodiments, the energy storage module 222 may have its own power source and/or battery pack. In other embodiments, the energy storage module 222 may pull power from the power source 232. In still further embodiments, the energy storage module 222 may include one or more capacitors 234. The one or more capacitors 234 may store an electrical charge, which may be administered to the patient. The processor 202 may be communicatively coupled to the energy storage module 222 to trigger the amount and timing of electrical energy to provide to the defibrillation port 208 and, subsequently, the patient 102.

In some embodiments, the defibrillator 108 may include a discharge circuit 236. The discharge circuit 236 may control the energy stored in the energy storage module 222. For example, the discharge circuit 236 may either electrical couple or decouple the energy storage module 222 to the defibrillation port 208. The discharge circuit 236 may be communicatively coupled to the processor 202 to control when the energy storage module 222 and the defibrillation port 208 should or should not be coupled to either administer or prevent a charge from emitting from the defibrillator 108. In some embodiments, the discharge circuit 236 may include on or more switches 238. In further embodiments, the one or more switches 238 may include an H-bridge.

In some embodiments, the defibrillator 108 may include a communication module 216. The communication module 216 may establish one or more communication links with either local hardware and/or software to the WCD system 104 and defibrillator 108 or to remote hardwire separate from the WCD system 104. In some embodiments, the communication module 216 may include one or more antennas, processors, and the like. The communication module 216 may communicate wirelessly via radio frequency, electromagnetics, local area networks (LAN), wide area networks (WAN), virtual private networks (VPN), RFID, Bluetooth, cellular networks, and the like. The communication module 216 may facilitate communication of data and commands such as patient data, episode information, therapy attempted, CPR performance, system data, environmental data, and so on.

In some embodiments, the processor 202 may execute one or more modules. For example, the processor 202 may execute a detection module 240 and/or an action module 242. The detection module 240 may be a logic device or algorithm to determine if any or a variety of thresholds are exceeded which may require action of the defibrillator 108. For example, the detection module 240 may receive and interpret all of the signals from the ECG port 210, the defibrillation port 208, the monitoring device 220, an external monitoring device, and the like. The detection module 240 may process the information to ensure the patient is still conscious and healthy. If any parameter indicates the patient 102 may be experiencing distress or indicating a cardiac episode, the detection module 240 may activate the action module 242.

The action module 242 may receive data from the detection module 240 and perform a series of actions. For example, an episode may merely be a loss of battery power at the power source 232 or the energy storage module 222, or one or more electrodes (e.g., ECG electrodes, defibrillation electrodes) may have lost connection. In such instances, the action module 242 may trigger an alert to the patient or to an outside source of the present situation. This may include activating an alert module. If an episode is a health risk, such as a cardiac event, the action module 242 may begin a series of steps. This may include issuing a warning to the patient, issuing a warning to a third party, priming the energy storage module 222 for defibrillation, releasing one or more conductive fluids proximate defibrillation electrodes 114, 116, and the like.

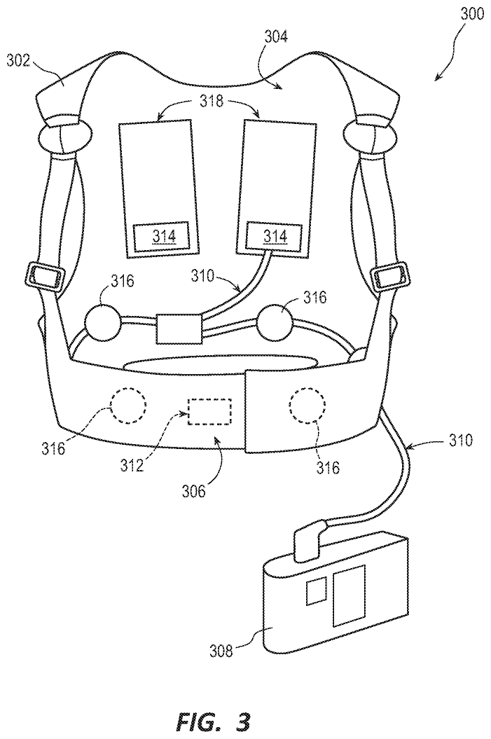

FIG. 3 is a diagram of sample embodiments of components of a WCD system 300 according to exemplary embodiments. The WCD system 300 may be an example of the WCD system 104 describe with reference to FIG. 1. In some embodiments, the WCD system 300 may include a support structure 302 comprising a vest-like wearable garment. In some embodiments, the support structure 302 has a back side 304, and a front side 306 that closes in front of a chest of the patient.

In some embodiments, the WCD system 300 may also include an external defibrillator 308. The external defibrillator 308 may be an example of the defibrillator 108 describe with reference to FIGS. 1 and 2. As illustrated, FIG. 3 does not show any support for the external defibrillator 308, but as discussed, the defibrillator 308 may be carried in a purse, on a belt, by a strap over the shoulder, and the like as discussed previously. One or more wires 310 may connect the external defibrillator 308 to one or more electrodes 312, 314, 316. Of the connected electrodes, electrodes 312, 314 are defibrillation electrodes, and electrodes 316 are ECG sensing electrodes.

The support structure 302 is worn by the patient to maintain electrodes 312, 314, 316 on a body of the patient. For example, the back-defibrillation electrodes 314 are maintained in pockets 318. In some embodiments, the inside of the pockets 318 may comprise loose netting, so that the electrodes 314 can contact the back of the patient. In some instances, a conductive fluid may be deployed to increase connectivity. Additionally, in some embodiments, sensing electrodes 316 are maintained in positions that surround the patient's torso, for sensing ECG signals and/or the impedance of the patient.

In some instances, the ECG signals in a WCD system 300 may comprise too much electrical noise to be useful. To ameliorate the problem, multiple ECG sensing electrodes 316 are provided, for presenting many options to the processor (202. The multiple ECG sensing electrodes 316 provide different vectors for sensing the ECG signal of the patient.

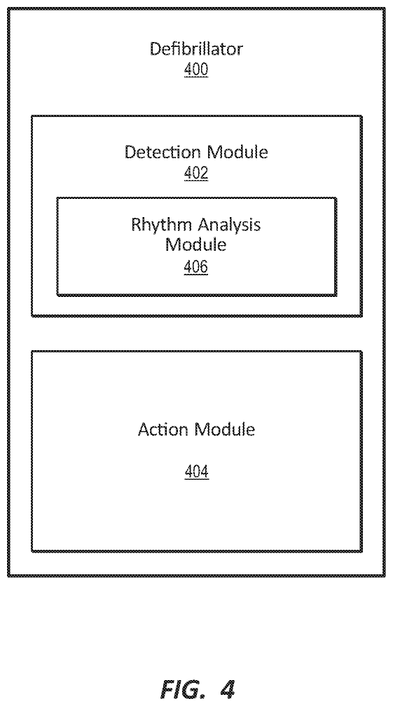

FIG. 4 is a block diagram illustrating components of one example of a defibrillator 400. The defibrillator 400 may be an example of the defibrillator 108 described with reference to FIGS. 1 and 2 and defibrillator 308 described with reference to FIG. 3. In this example, the defibrillator 400 has detection module 402 and an alert module 404, and a data storage module 406. In some embodiments, the detection module 402 may include a rhythm analysis module 406.

The detection module 402 may be an example of the detection module 240 described with reference to FIG. 2. For example, the detection module may receive and interpret signals received from the ECG port, defibrillation port, an external monitoring device, and the like. The detection module 402 may process all of the data to determine a status of the patient. For example, the detection module 402 may determine if the patient is healthy and conscious. The detection module 402 may also analyze the data for a shockable rhythm or any other irregularities.

In some embodiments, the rhythm analysis module 406 may analyze the signal from the ECG port to determine if the patient is experiencing a regular heart rate, VT, SVT, or another condition. One method of determining the difference between VT and SVT heart rates is disclosed in U.S. patent application Ser. No. 16/380,037 filed on Apr. 10, 2019 the disclosure of which is hereby incorporated by reference in its entirety.

Filtering heart rate data may provide a long-term heart rate of a patient as well as maximum and minimum data trends. However, merely knowing a long-term heart rate trend does not provide physicians with the knowledge of the frequency or occurrence of VT or SVT beats. Knowing the heart rate trend along with VT and SVT data provides physicians with more information to treat the patient. Patients with sustained VT and SVT can be life threatening and require different care than abnormal heart rhythms.

In some embodiments, the rhythm analysis module 406 may distinguish between VT and SVT beats. In some instances, the rhythm analysis module 406 may use an estimated QRS width to distinguish between VT and SVT beats. For example, ventricular beats may be wider than SVT beats and typically beats wider than 120 milliseconds may be considered wide. However, some people have wide complexes for a normal, super-ventricular rhythm. Therefore, in some embodiments, a template of a normal QRS complex for the patient is utilized to differentiate between VT and SVT widths.

Once the rhythm analysis module 406 has a template and/or method to differentiate between VT and SVT beats, the rhythm analysis module 406 may track separate heart rate trend buffers for each type of beat. For example, the rhythm analysis module 406 may store the maximum, minimum, and average rate value once every hour the WCD is worn by the patient for both VT and SVT beats. In some embodiments, the rhythm analysis module 406 may measure the heart rate at least once a minute and in some instances every two to five seconds. Each measurement may be associated with a segment of time. Each heart rate measurement may have a QRS width associated with it. This QRS may enable the rhythm analysis module 406 to classify the beat as a ventricular rate or supra-ventricular rate.

Once the beats are classified, the rhythm analysis module 406 may track the separate maximum, minimum, and average statistics for ventricular and supra-ventricular beats. The rhythm analysis module 406 may also determine a quantity of ventricular beats that occurred in a given time period. The rhythm analysis module 406 may also determine how long a VT episode lasted. Furthermore, the percentage of VT and SVT beats for a given time period may also be determined. Every beat the rhythm analysis module 406 identifies has a separately tracked QRS width associated with it.

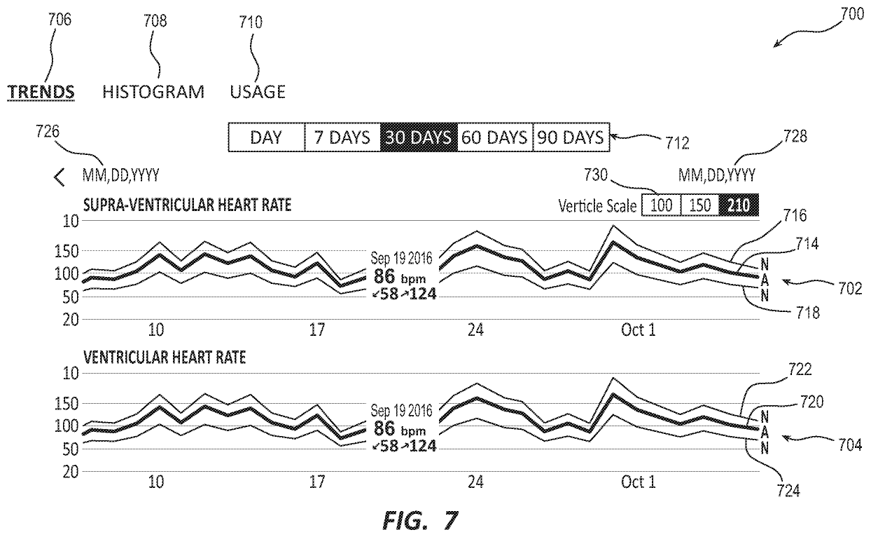

Once the rhythm analysis module 406 has determined and categorized the VT and SVT beats, the rhythm analysis module 406 may illustrate the different types of beats. In one embodiment, shown in FIG. 7, the rhythm analysis module 406 may display SVT beats and VT heartrate in two separate graphs, 702 and 704 respectively. For example, the display 700 may include the trends 706 for different time periods 708. For example, the display may toggle between a day, a week, a month, 2 months, 90 days, or some other time period, which may be customizable. In some embodiments, the display 700 may toggle between trends 706, histogram 708, usage 710, or another set of data. The SVT segment graph 702 may include an average heart rate as well an upper average limit 716 and a lower average limit 718. Similarly, the VT segment graph 704 may include an average 720 and an upper average limit 722 and a lower average limit 724. In some embodiments, the display 700 may show a beginning date 726 and an ending date 728 for the time period displayed in the graphed 702, 704. In some embodiments, the display 700 may include a vertical graph toggle 730 which may change the scale of the vertical display. In another embodiment, the graphical representation may have a toggle button (not shown) which may enable a user to view only one of the VT and SVT graphs 702, 704 at a time.

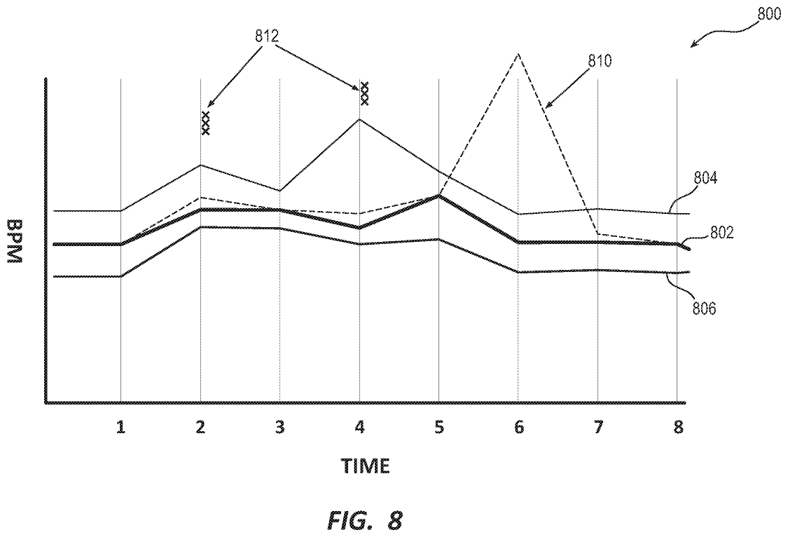

As shown in FIG. 8, the rhythm analysis module 506 may alternatively or additionally generate a display 800 a single heart rate average 802 with separate error bars for ventricular and supra-ventricular segments. The display also shows the atrial maximum BPMs 804 and the atrial minimum BPMs 806. The display also includes the ventricular beat maximum 810 as well as ventricular outliers 812. In some embodiments, VT beats may not be detected, and the dotted line would not be displayed.

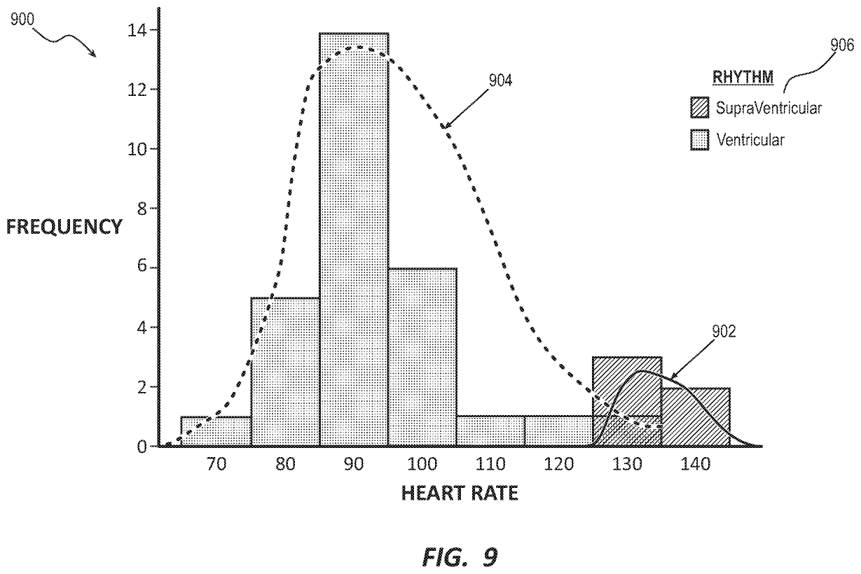

As shown in FIG. 9, the rhythm analysis module 506 may additionally or alternatively generate a bar chart 900 to display histogram of heart rhythm analysis. The key 906 may be shown which bars relate to either VT or SVT beats. In some embodiments, the graph 900 may include a probability distribution for VT 902 and SVT 904 beats. The graph 900 may be static or may be interactive. The user maybe able to select a section of the graph and cause another plot to pop up, or it could generate a data summary. The graph 900 could also include an interactive slider to set the time window of interest.

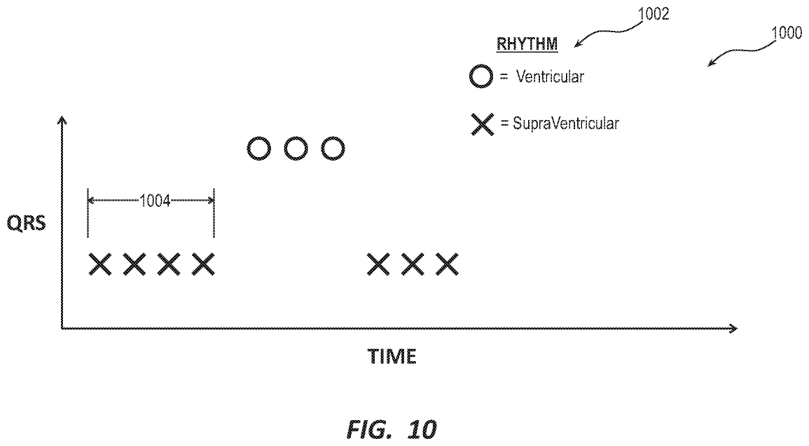

In some embodiments, as shown in FIG. 10, the rhythm analysis module 506 may additionally or alternatively generate a graph 1000 displaying QRS widths for VT and SVT beats. The graph 1000 may include a key 1002 which, in this example, shows the circles represent VT beats and the X's represent SVT beats. The graph 1000 may display a width 1004 of each beat. The width of the beat as shown visibly may enable the physician to determine why beats are classified as SVT and VT beats. The width 1004 of the beat may also enable the physician to visibly see the width of the beats, the consistency of the width of the beats and perhaps if more testing or analysis is required to further investigate the beats and the patient's condition.

Referring back to FIG. 4, the action module 404 may be one example of the action module 242 described with reference to FIG. 2. For example, the action module 404 may receive data from the detection module 402 and perform a series of actions. For example, the detection module 402 may detect an irregular heartbeat or a cardiac event happening and may relay information to the action module 404 which may initiate a series of actions. This may include issuing a warning to the patient, issuing a warning to a third party, priming an energy storage module for defibrillation, releasing one or more conductive fluids proximate defibrillation electrodes, and the like. In other embodiments, the event may be a device event which may indicate one or more issues with the WCD. For example, there may be a loss of battery power or an electrode may have a bad connection.

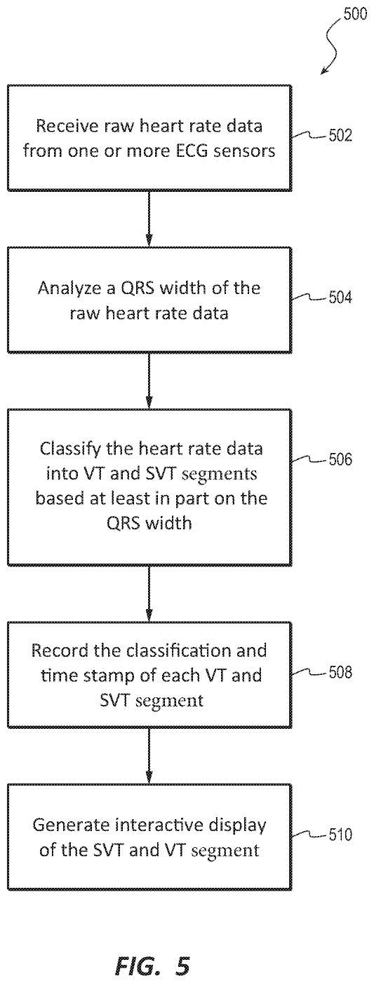

FIG. 5 is a flow chart illustrating an example of a method 500 for WCD systems, in accordance with various aspects of the present disclosure. For clarity, the method 500 is described below with reference to aspects of one or more of the systems described herein.

At block 502, the method 500 may include receiving raw heart rate data from one or more ECG sensors. Then, at block 504, the method 500 may include analyzing a QRS width of the raw heart rate data. The QRS width may provide a way for the method to categorize the different types of heart beats. In some embodiments, the method 500 may have to filter out noise from the signal before analyzing QRS widths. The method 500 may be able to differentiate from VT and SVT segments because VT segments are generally wider than SVT segments. Therefore, at block 506, the method 500 may include classifying the segments into VT and SVT segments based at least in part on the QRS width. Once the method 500 distinguishes the segments, the method 500 may keep separate heart rate tend buffers for each type of beat. For example, the method 500 may store a maximum, minimum, and average heart rate value at least one a minute and, in some embodiments, as often as every 2.4 seconds.

At block 508, the method 500 may record the classification and time stamp of each VT and SVT segment. This may associate each measurement with a segment of time. At block 510, the method may generate interactive displays of the SVT and VT segments. This may include displaying the number of VT occurred in a given time period, or how long a VT episode lasted. In further embodiments, the method 500 may display the percentage of VT and SVT beats for a given time period. The displays may include the maximum, minimum, and average heart rates over a period of time. The period of time may be predetermined or may be customizable. In some embodiments, the heart rate trends may be displayed alongside other data such as activity level or movement of the patient. This may enable a physician to determine how a patient's activity level is affecting their heart health.

Thus, the method 500 may provide for categorizing and graphing WCD data. It should be noted that the method 500 is just one implementation and that the operations of the method 500 may be rearranged or otherwise modified such that other implementations are possible.

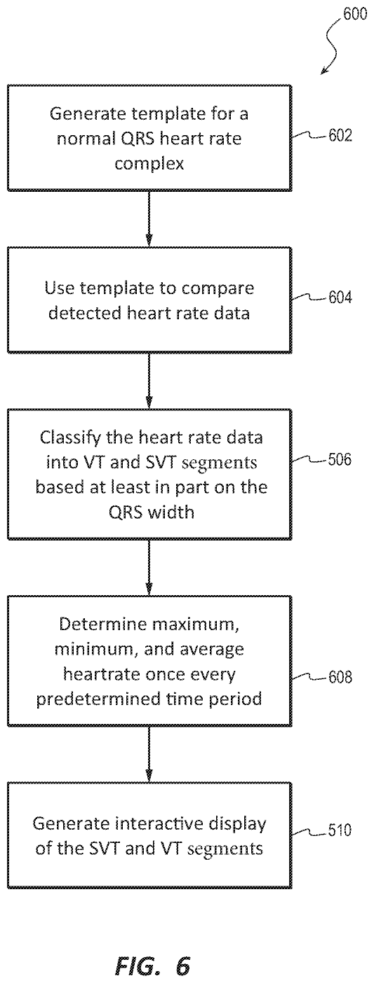

FIG. 6 is a flow chart illustrating an example of a method 600 for WCD systems, in accordance with various aspects of the present disclosure. For clarity, the method 600 is described below with reference to aspects of one or more of the systems described herein.

At block 602, the method 600 may include generating a template for a normal QRS heart rate complex for a given patient. This may assume that a patient's normal beat is a VT, then any beat that varies from the normal beat is an SVT beat. Using a template may potentially distinguish between VT and SVT beats even for a patient who has normally wide beats.

At block 604, the method 600 may use the template to compare to the detected heart rate data. Then, at block 506, the method 600 may classify the segment data into VT and SVT beats base data least in part on the QRS width. For example, in some embodiments, the method 600 may use a template to help distinguish VT from SVT. If the detected complex morphology matches the template, the method 600 may determine a normal beat is present and classify the beat as SVT. If the detected complex does not match the template, the method 600 may assume it is an abnormal beat and classify the beat as VT. Then, at block 608, the method 600 may determine a maximum, minimum, and average heartrate once every predetermined time period. The predetermined time period may be once a minute. In other embodiments, the predetermined time period may be less than one minute and may occur once every 2.4 seconds. The method 600 may then, at block 510, generate an interactive display of the SVT and VT segments.

Thus, the method 600 may provide for categorizing and graphing WCD data. It should be noted that the method 600 is just one implementation and that the operations of the method 600 may be rearranged or otherwise modified such that other implementations are possible.

A person skilled in the art will be able to practice the present invention after careful review of this description, which is to be taken as a whole. Details have been included to provide a thorough understanding. In other instances, well-known aspects have not been described, in order to not obscure unnecessarily this description.

Some technologies or techniques described in this document may be known. Even then, however, it is not known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, component or process that are identical or at least similar or related. Where made, such a further effort was not required, but was nevertheless made gratuitously so as to accelerate comprehension by the reader. Even where made in this document, such a further effort might not have been made completely consistently for all of the versions or embodiments that are made possible by this description. Accordingly, the description controls in defining an item, aspect, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

What is claimed is:

1. A method to display patient data collected by a wearable cardioverter defibrillator (WCD), the method comprising:
receiving raw heart rate data from one or more electrocardiogram (ECG) sensors;
classifying the heart rate data into one of a ventricular (VT) and supra-ventricular (SVT) segment;
recording the classification and time stamp of each VT and SVT segment; and
generating one or more interactive displays of each VT and SVT segments.

2. The method of claim 1, wherein classifying the heart rate data further comprises:
generating a template for a normal QRS heart rate complex for a user of the WCD; and
comparing the template to the QRS width of the raw heart rate data.

3. The method of claim 2, further comprising:
generating one or more graphs of VT and SVT segments displaying a QRS width of each VT and SVT heartbeat.

4. The method of claim 1, further comprising:
determining at least one of a maximum, a minimum, an average, and some combination thereof of the VT and SVT segments once every predetermined time period.

5. The method of claim 1, further comprising:
determining a percentage of segments that are VT segments and a percentage of segments that are SVT segments for a predetermined time period; and
generating a visual representation to visibly display the percentage of VT segments and SVT segments for the predetermined time period.

6. The method of claim 5, wherein the predetermined time period is adjustable.

7. The method of claim 1, further comprising:
generating a graph of VT segments for a predetermined time period;
generating a graph of SVT segments for the predetermined time period; and
combining the graphs into a display.

8. The method of claim 1, further comprising:
receiving movement data from a motion sensor coupled to the WCD;
analyzing movement data to determine patient mobility data;
generating a graph of the patient mobility data; and
displaying the graph of patient mobility data with the VT and SVT segment data.

9. The method of claim 1, further comprising:
generating a maximum, minimum, and average segment reading for a specific time stamp; and
displaying the maximum, minimum, and average segment for the specific time stamp when prompted.

10. The method of claim 1, further including:
analyzing a QRS width of the raw heart rate data.

11. A wearable cardiac defibrillator (WCD) system for monitoring health of a patient wearing the WCD system, the system comprising:
at least one sensor positioned to gather data about the patient;
one or more memories, the one or more memories configured to analyze patient data; and
one or more processors configured to cause the system to:
receive raw heart rate data from one or more electrocardiogram (ECG) sensors;
classify the heart rate data into one of a ventricular (VT) and supra-ventricular (SVT) segment based at least in part on the QRS width;
record the classification and time stamp of each VT and SVT segment; and
generate one or more interactive displays of each of the VT and SVT segment.

12. The WCD system of claim 11, wherein when classifying the heart rate data, the processor is further configured to:
generate a template for a normal QRS heart rate complex for a user of the WCD; and
compare the template to the QRS width of the raw heart rate data.

13. The WCD system of claim 12, wherein the processor is further configured to:
generate a graph of VT and SVT segments displaying a QRS width of each VT and SVT segment.

14. The WCD system of claim 11, wherein the processor is further configured to:
determine at least one of a maximum, a minimum, an average, and some combination thereof of the VT and SVT segment once every predetermined time period.

15. The WCD system of claim 11, wherein the processor is further configured to:
determine a percentage of segments that are VT segments and a percentage of segments that are SVT segments for a predetermined time period; and
generate a visual representation to visibly display the percentage of VT segments and SVT segments for the predetermined time period.

16. The WCD system of claim 15, wherein the predetermined time period is adjustable.

17. The WCD system of claim 11, wherein the processor is further configured to:
generate a graph of VT segments for a predetermined time period;
generate a graph of SVT segments for the predetermined time period; and
combine the graphs into a display.

18. The WCD system of claim 11, wherein the processor is further configured to:
receive movement data from a motion sensor coupled to the WCD;
analyze movement data to determine patient mobility data;
generate a graph of the patient mobility data; and
display the graph of patient mobility data with the VT and SVT segment data.

19. The WCD system of claim 11, wherein the processor is further configured to:
generate a maximum, minimum, and average heart rate reading for a specific time stamp; and
display the maximum, minimum, and average heart rate for the specific time stamp when prompted.

20. A method to display data collected by a wearable cardioverter defibrillator (WCD), the method comprising:
positioning at least one electrocardiogram (ECG) sensing electrodes to measure electrical activity of a heart of a person;
receiving at least one ECG signal from the at least one ECG electrodes;
analyzing the signal into usable data including a QRS width of each segment;
classifying the heart rate data into one of a ventricular (VT) and supra-ventricular (SVT) segment based at least in part on the QRS width;
recording the classification and time stamp of each VT and SVT segment; and
generating one or more interactive displays of the VT and SVT segments.
